(19) United States
(12) Patent Application Publication
(10) Pub. No.: US 2008/0269822 A1
(43) Pub. Date: Oct. 30, 2008

(54) Device for Evaluating Positions of an Implantable Medical Device

(76) Inventors: Karin Ljungstrom, Hasselby (SE); Anders Bjorling, Jarfalla (SE); Hans Strandberg, Sundbyberg (SE)

Correspondence Address: SCHIFF HARDIN, LLP;PATENT DEPARTMENT, 6600 SEARS TOWER, CHICAGO, IL, 60606-6473, US

(21) Appl. No.: 11/666,960

(22) PCT Filed: Nov. 2, 2004
(86) PCT No.: PCT/SE04/01600

§ 371 Date: Nov. 12, 2007

(51) Int. Cl. A61N 1/368

(52) U.S. Cl. 607/18

(57) ABSTRACT

In a device and method for evaluating positions of a medical lead during an implantation procedure, an IEGM signal and a signal indicative of heart pumping activity are obtained for each of a number of different lead positions, and those signals are stored dependent on the different lead positions. A processor automatically determines a lead position, from among the stored lead positions, that results in most favorable hemodynamics of the heart, based on the IEGM signal and the pumping activity signal.

### BACKGROUND OF THE INVENTION

1. Field of the Invention

The present invention generally relates to implantable medical devices such as implantable cardiac pacemakers and in particular to a device and a method for obtaining information related to the heart pumping activity at different lead positions within a heart and for using the information to evaluate the different lead positions in order to identify the optimal position with respect to the heart pumping activity.

2. Description of the Prior Art

The technology of cardiac pacemakers has developed in sophistication and functionality over the years. In general, cardiac pacemakers are designed to control the heart by correcting or compensating for various heart abnormalities which can be encountered in human patients. For example, cardiac pacemakers may provide therapeutic stimulation to the heart by delivering therapeutic pulses such as pacing, cardioversion or defibrillation pulses.

Commonly, the pulses are delivered to the heart via electrodes disposed on implantable leads coupled to the pacemaker. The pacemaker performs various sensing and pulsing functions by receiving and delivering signals through the leads. The placements of the electrodes, i.e. the leads, with respect to one or more cardiac locations is such so that the desired electrical functions such as stimulation or heart signal sensing are ensured. For example, the leads may position the electrodes with respect to one or more cardiac locations so that the pacemaker can deliver pulses to the appropriate locations of the heart.

Leads may be placed in one or more of a variety of different cardiac locations. In particular, the placement of the leads may be dependent on the cardiac conditions of the patient and the therapy to be delivered.

A proper placement of the leads, i.e. the electrodes, is essential because both the desired electrical functions, i.e. stimulation or heart signal sensing, and the desired heart muscle reaction (activity propagation) are dependent on the position of the lead. However, it is often difficult to determine whether a lead has been properly positioned and adequate tissue contact has been achieved. Today, pacemaker leads are normally checked during the implantation to ensure satisfactory electrical performance by use of a PSA (pacemaker system analyzer). A PSA is an external equipment connected to the implanted leads. In general three electrode and lead properties are checked by use of a PSA to be satisfactory;
1. Stimulation threshold. That the required electrical stimulation energy to activate the heart is low enough.
2. Heart signal. That the spontaneous heart signal picked up by the lead system is of enough amplitude.
3. Lead impedance. That the conductive path of the lead(s) together with body fluids and tissue is in physical good condition.

Furthermore, it is often difficult to get the lead to the proper or desired site, specially the left ventricular lead through the coronary sinus, partly due to technical difficulties and anatomic differences, but also due to the lack of a standard procedure for identifying the optimal or best stimulation spot for that specific patient. One method for left ventricular lead placement includes so called venogram, where a fluoro-visible dye is injected into the veins so that the veins are visible using a fluoroscopic device.

Thus, there is a need of an improved device and method that, in an efficient way, is capable of identifying the optimal position of the lead or the leads within a heart with respect to the heart pumping activity during an implantation procedure of an implantable medical device.

### SUMMARY OF THE INVENTION

Thus, an object of the present invention is to provide a simplified and improved system and method for finding the optimal lead placement for one or more leads with respect to heart pumping activity during an implantation procedure.

In the context of the present invention, the term “mechanical sensor” relates to sensors capable of providing one or more signals indicative of cardiac mechanical activity, such as acoustic sensors, accelerometer-based sensors, pressure sensors, etc.

According to the present invention, there is provided a device for evaluating positions of a medical lead during an implantation procedure. The medical lead has at least one electrode for stimulating and sensing, and at least one mechanical sensor, the sensor being arranged to provide signals related to heart pumping activity. The device has a measuring unit that records sensor signals at different lead positions; storage unit that stores the sensor signals; and a processor that determines the lead position resulting in the most favorable hemodynamics of heart based on a measure of the heart activity at each position.

The above object is also achieved in accordance with the present invention by a method for evaluating positions of a medical lead during an implantation procedure, wherein the medical lead has at least one electrode for stimulating and sensing, and at least one mechanical sensor, which sensor is arranged to provide signals related to heart pumping activity. The method includes recording sensor signals at different lead positions; storing the sensor signals; and determining the lead position resulting in the most favorable hemodynamics of heart based on a measure of the heart activity at each position.

The above object also is achieved in accordance with the present invention by a computer readable medium encoded with a data structure representing instructions for causing a programmable device to perform the method according to the invention.

Thus, the invention is based on the idea of utilizing signals characteristic of the heart pumping activity to evaluate different positions of a medical lead or medical leads during an implantation procedure in order to identity the optimal site or placement for the lead or the leads with respect to a desired heart muscle reaction. That is, physiologic parameters reflecting hemodynamic performance are derived and evaluated for each lead position in order to determine the optimal site.

This solution provides several advantages over the existing solutions. One advantage is that the optimal site for placing the lead (or the leads) can be accurately determined with respect to anatomic differences of a specific patient, the specific therapy to be delivered and the desired heart muscle reaction (activity propagation).

Another advantage is that the lead site optimization is incorporated into the implant procedure. This provides for significant time savings in comparison with prior art solutions, which, in turn, reduces the risk for infection because the time required for the implantation procedure is decreased.

In a preferred embodiment of the present invention, the lead or the leads incorporate one or more pressure sensors. The pressure sensors transduce hemodynamic pressure variations, for example, left ventricular pressure and/or right ventricular pressure, so as to provide one or more signals indicative of the heart pumping activity, which signals are used to determine, for example, a measure of the heart pumping activity such as pre-ejection time, left ventricular ejection time, or the ventricular contractility (peak left ventricular pressure change during systole or LV+dp/dt).

In accordance with another preferred embodiment of the present invention, the lead or the leads incorporate one or more accelerometer-based sensors, for example, cardiac wall motion sensors. The cardiac wall motion sensors transduce accelerations of cardiac tissue to which the leads are attached, so as to provide one or more signals indicative of cardiac mechanical activity, which can be used to derive physiologic parameters indicative of cardiac performance, including stroke volume, contractility, pre-ejection period, and ejection time, which can be used as a measure of heart pumping activity.

According to an embodiment of the present invention, the lead position resulting in the shortest pre-ejection time period (PEP) is determined to be the lead position resulting in the most favorable hemodynamics of the heart, wherein PEP is determined as the period from the onset of a QRS or an emitted ventricular stimulation until the opening of the aortic valve. The opening of the aortic valve is determined to the moment when a maximum of the time derivative of the ventricular pressure (i.e. dp/dt) occurs after the onset of a QRS complex or an emitted ventricular stimulation. In one embodiment, a pressure sensor is placed tranvenously through the coronary sinus and located in the coronary vein in order to sense the left ventricular pressure.

According to another embodiment of the present invention, the lead position resulting in the lowest value of the quotient between PEP and left ventricular ejection time (LVET) is determined to be the lead position resulting in the most favorable hemodynamics of the heart. LVET is affected by the contractility of the myocardium and by outflow obstructions at the left ventricle. At a degraded contractility with a low stroke volume LVET will decrease, whilst it will be lengthen at outflow obstructions, such as aortic stenosis, and at a large central stroke volume. PEP tends to increase at, inter alia, cardiac insufficiency. As with PEP, LVET must be corrected with respect to the heart rate. The PEP/LVET quotient reflects the function of the left ventricle in a more efficient way than the individual components and is not dependent on the heart rate. Hence, the quotient PEP/LVET provides an efficient and reliable measure of the heart pumping activity.

According to other embodiments of the present invention, the co-ordination of the contraction of the left ventricle and the right ventricle, respectively, or, in other words, the timing difference or delay between the contraction of the left ventricle and the right ventricle, respectively, is recorded at each lead position and the lead position resulting in the shortest delay between the contraction of the left ventricle and the contraction of the right ventricle is determined to be the lead position resulting in the most favorable hemodynamics of the heart. Preferably, the pressure in the coronary vein, which pressure is proportional to the left ventricular pressure, and the right ventricular pressure are sensed. Alternatively, the proportionality between the left ventricular pressure and the right ventricular pressure is utilized when optimizing the lead position or the lead positions.

According to still another embodiment of the present invention, the ventricular contractility (peak left ventricular pressure change during systole or LV+dp/dt) is recorded at each lead position and the lead position resulting in the highest LV+dp/dt is determined to be the lead position resulting in the most favorable hemodynamics of the heart. Because the left ventricle systolic performance directly determines the ability of the heart to pump blood through the systemic circulation, it has been found that an efficient way to assess the heart activity is to examine how well the left ventricle contracts in order to determine the effectiveness of the left ventricle as a pump. Therefore, by using the left ventricle contraction effectiveness or “contractility” as the measure of the heart pumping activity, the lead position can be optimized in an efficient and reliable way.

According to a further embodiment of the present invention, the cardiac output is determined at each lead position and the lead position resulting in the highest cardiac output, which is the volume of blood in liters ejected by the heart per minute, is determined as the lead position resulting in the most favorable hemodynamics of the heart.

Preferably, the AV interval between stimulation of the atrium and the ventricle and/or the VV interval between stimulation of the right and left ventricles are optimized before the measurement related to the determination of the performance of the actual lead position is performed.

As realized by the person skilled in the art, the method of the present invention, as well as preferred embodiments thereof, are suitable to realize or implement as a computer program or a computer readable medium, preferably within the contents of a device in accordance with the first aspect of the present invention and in particular within the processing means of such a device.

Further objects and advantages of the present invention will be discussed below by means of exemplifying embodiments.

### BRIEF DESCRIPTION OF THE DRAWINGS

FIG. 1 is a schematic diagram of a device for evaluating positions of a medical lead during an implantation according to an embodiment of the present invention.

FIG. 2 is schematic diagram of a Graphical User Interface which can be presented on a display of the device shown in FIG. 1 according to an embodiment of the present invention.

FIG. 3 is a flow chart describing the principle steps of the process of evaluating different position of a lead of an implantable medical device to find an optimal lead site in accordance with the present invention.

### DESCRIPTION OF THE PREFERRED EMBODIMENTS

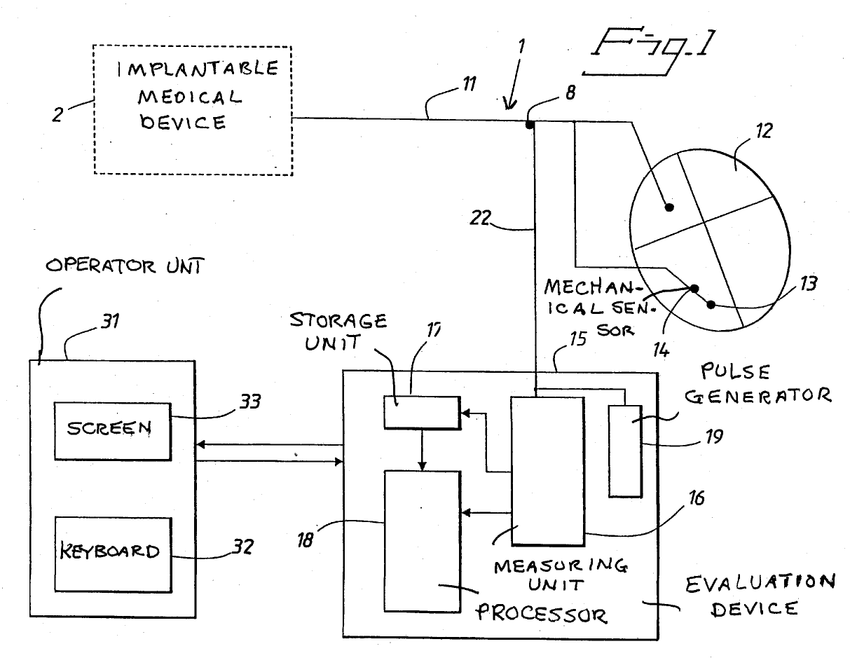

FIG. 1 is a diagram illustrating an implantable medical device (IMD) and a device for evaluating positions of a medical lead during an implantation procedure in order to find an optimal lead placement according to an embodiment of the present invention. IMD 1 comprises a lead 11, which may be unipolar or bipolar, and may be adapted to operate in cooperation with a wide variety of implantable medical devices.

In FIG. 1, one position of lead 11 in the heart 12 of a patient is shown. The lead 11 may include any of the passive or active fixation mechanisms known in the art for fixation of the lead to the cardiac tissue when final position has been found. As an example, lead distal tip (not shown) may include a tined tip or a fixation helix. The lead 11 carries one or more electrodes 13, such as a tip electrode or a ring electrode. The electrode 13 senses electrical signals associated with depolarization and repolarization of the heart 12, and thereby can IEGM signals be obtained. In addition, the electrode 13 may also transmit pacing pulses for causing depolarization of cardiac tissue adjacent to the electrode 13. Furthermore, the lead 11 also carries a sensor 14 arranged to sense signals related to the pumping activity of the heart 12. The sensor 14 may include different types of mechanical sensors that can be used with the present invention will be discussed below.

The IMD 1 is connectable to an active implantable medical device 2, for example, a pacemaker and after completion of the evaluation procedure and after positioning of the at least one electrode 13 at the final position, the IMD 1 is connected to the active implantable medical device 2. The active implantable medical device 2 comprises a pacemaker circuit (not shown), a processor (not shown), coupled to the at least one electrode 13, and a memory circuit (not shown) coupled to the processor. The pacemaker circuit comprises, among other things, a pulse generator circuit, a timer and control circuit coupled to the pulse generator circuit and the processor, and a telemetry circuit. The telemetry circuit, which when implanted telemetrically communicates with an external programmer, is coupled within the pacemaker to the memory circuit and the timing and control circuit. The operation and function of the different circuits of the IMD 2 is well-known for the man skilled in the art for what reason they are not described in further detail in the context of this application.

The evaluation device 15 includes a measuring unit 16, a storage unit 17, a processor 18, and a pulse generator 19. The storage unit 17 may include a random access memory (RAM) and/or a non-volatile memory such as read-only memory (ROM): As will be appreciated by one of ordinary skill in the art, storage means may include various types of physical devices for temporary and/or persistent storage of data which includes solid state, magnetic, optical and combination devices. For example, the storage means may be implemented using one or more physical devices such as DRAM, PROMS, EPROMS, EEPROMS, flash memory, and the like. The processor 18 may be a microprocessor and may also include storage capacity, which may include a random access memory (RAM) and/or a nonvolatile memory such as read-only memory (ROM). The pulse generator 19 is connected to the lead 11 and is controlled by the processing means 18 and arranged to generate stimulation pulses for deliverance to the cardiac tissue adjacent to the electrode 13 of the lead 11.

According to an embodiment, the evaluation device 15 is detachably connected to the lead 11 during the implantation procedure at a connecting terminal 8 of the lead 11. The connecting terminal 8 is preferably adapted to the guide wire used during the implantation to place the lead 11 at different positions within the heart 12. In the embodiment described above and shown in FIG. 1, the measuring unit 16 is connected to the lead 11 or to the leads via the connection terminal 8 and a connection lead 22.

In an alternative embodiment, the device 15 is provided with one or more leads 11. The lead 11 comprises one or more electrodes 13, such as a tip electrode or a ring electrode. The electrode 13 senses electrical signals associated with depolarization and repolarization of the heart 12. In addition, the electrode 13 may also transmit pacing pulses for causing depolarization of cardiac tissue adjacent to the electrode 13. Furthermore, the lead 11 also carries a sensor 14 arranged to sense a signals related to the pumping activity of the heart 12. The sensor 14 may include different types of sensors that can be used with the present invention as will be discussed below.

The measuring unit 16 is arranged to measure or record the sensor signals and signals characteristic of the heart pumping activity at the current lead position by means of the sensor 14 and/or the electrode 13 when the evaluation device 15 is connected directly to the lead 11 or the leads at the connection terminal 8 (or terminals if the are more than one lead) via the connection lead 22. Furthermore, the sensor signals and/or signals characteristic of the heart activity are stored in the storage means 17. The processor 18 is connected to the measurement unit 16 and is arranged, inter alia, to determine a measure of the heart pumping activity resulting from a stimulation pulse delivered to the cardiac tissue as current lead position based on the signals measured or recorded by the measuring means 16. The procedure for determining the measure will be described in further detail below.

In addition, the evaluation device 15 comprises an operator unit 31 as can be seen in FIG. 1. The operator unit 31 comprises a keyboard 32, which allows the operator to input, for example, control commands, and a display or screen 33 for presenting information related to the evaluation of the obtained sensor signals from the different lead positions in or at heart 12. In an another embodiment, the screen 33 is a touch sensitive screen and the user can perform operations such as the above mentioned by touching the screen 33. The touch sensitive screen may include a number of soft-keys in order to present different commands at different presented interfaces on the screen 33. Of course, the touch sensitive screen can be combined with a keyboard and positioning device. According to an embodiment, storage means may be arranged and/or in the operator unit 31. The embodiment described above and shown in FIG. 1 can be integrated within a standard pacemaker system analyzer (PSA), which is connected to the lead or the leads of the medical device by adding a measuring means 16 and include adequate processing software.

Alternatively, the operator unit 31 may, as will be appreciated by those skilled in the art, be a stand-alone unit which is connectable to the evaluation device 15, the implantable medical device or the PSA. During the implantation procedure, the physician can, for example, use the keyboard 32 of the operator unit 31 to activate the pulse generator 19 to deliver a stimulation pulse when the lead has been placed on a site which is to be evaluated. In case of a touch sensitive screen, the pulse generator can be activated using, for example, a soft-key displayed on the screen 33.

Of course, there are a plurality of other conceivable embodiments, and the above described embodiments should be considered only as exemplifications. For example, the operator unit 31 may be a personal computer or a laptop computer.

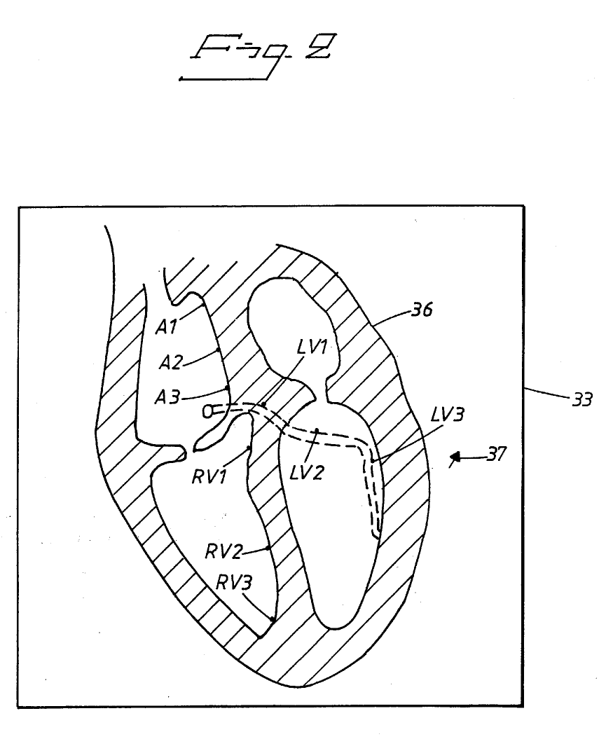

Turning now to FIG. 2, a Graphically User Interface which can be presented on the screen 33 is shown. The operator unit 31 may be arranged to present a Graphical User Interface on the screen 33 containing a diagrammatic picture 36 of the heart 12 of the patient in which the IMD 1 and the lead or the leads are about to implanted into. The user can perform operations on the picture of the heart 36 such as rotating the heart or enlarging or reducing parts thereof. These operations can, for example, be performed by means of the keyboard 32 or a positioning device such as a mouse. As mentioned above, the screen 33 may be a touch sensitive screen and the user can perform operations such as the above mentioned by touching the screen 33 and the touch sensitive screen may include a number of soft-keys in order to present different commands at different presented interfaces on the screen 33.

The physician or the attendant personnel can input the present position or positions for the lead or the leads, for example, in right atrium AI, A2, A3, in left ventricle LV1, LV2, LV3, and/or in right ventricle RV I, RV2, RV 3 on the screen 33 by entering the position data for the lead or the leads using the keyboard 32. Alternatively, the physician or the attendant personnel can by means of a cursor 37 controlled either by the keyboard 32 or a positioning device such as a mouse point out the present lead and electrode positions in right atrium A 1, A2, A3, in left ventricle LV 1, LV2, LV3, and/or in right ventricle RV1, RV2, RV 3 on the screen 33. Moreover, in case of a touch sensitive screen, the position or the positions can be input by touching the screen 33 at the proper position thereby indicating the present position or positions, for example, in right atrium A1, A2, A3, in left ventricle LV1, LV2, LV3, and in right ventricle RV 1, RV2, RV 3. The position information or data can be obtained by means of a number of different methods, for example, X-ray, or ultrasound. Alternatively, the physician may gain knowledge of the actual position by personal feeling from his or hers fingers during the implantation procedure. Furthermore, position data or information of each lead positions is also stored in the storage means of operator unit 31 or the storage means 17 the evaluation device 15, which can be performed either automatically at inputting the position data or manually using, for example, the keyboard 32. As will be discussed in more detail below, a measure of score value of the heart pumping activity is calculated or determined for each lead position and the measure or value is stored together with the position data for the corresponding position. Subsequently, when a measure or score has been determined for all lead positions, the processor 18 determines the lead position resulting in the most favorable hemodynamics of the heart or, in other words, the most efficient heart activity, as will be discussed in greater detail below. The measure for each lead position together with the lead position resulting in the most favorable hemodynamics of the heart can be presented for the user, for example, the physician, visually on the screen 33 of the operator unit 31. Thereby, the user can easily identify the best or optimal position or site for the lead with respect to the heart pumping activity in a clear way. These measures can, for example, be presented on the screen 33 by means of a graphical method such as color intensity or a bar diagram. For example, a high score or measure, i.e. a favorable hemodynamics of the heart, may be indicated with a dark color and a lower score with a paler color.

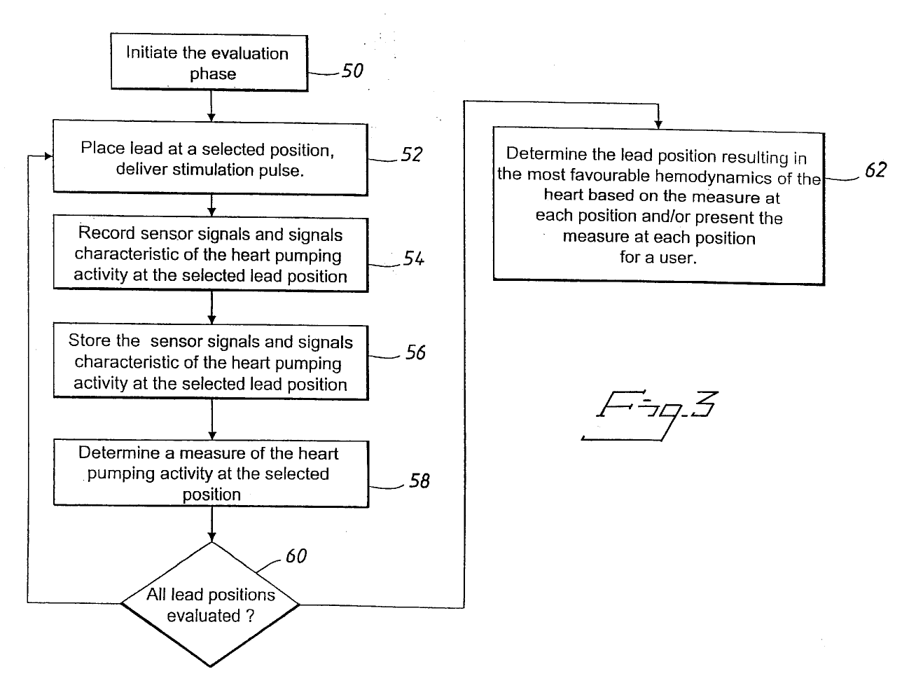

With reference now to FIG. 3, a flow chart of the principles of the process of evaluating different position of one or more leads according to the present invention during implantation of the medical device will be described. First, at step 50, a test phase is activated by the user. This can be performed by activating an evaluation or optimization sequence or program stored in the storage unit 17 of the evaluation device 15 or a storage unit of the operator unit 31 by a start command input by means of the keyboard 32. For example, an activation of the evaluation sequence may result in that the Graphical User Interface, as described above, is presented on the screen 33 containing a diagrammatic picture 36 of a heart. As discussed above, the position or the placement of the lead is essential for the functions of the medical device as well as regards to obtaining the desired heart muscle reaction and finding or identifying the optimal lead position with respect to the heart activity is often difficult. Therefore, a number of different lead positions may have to be tested and evaluated during the implantation procedure of the medical device in order to find a placement of the lead that gives the optimal heart activity at stimulation. Moreover, the sensor or sensors are positioned at the appropriate positions with respect to the signals required to obtain the specific measure utilized to evaluate the lead positions.

Then, at step 52, the physician or an attendant personal places the lead 11 at a first position by means of the guide wire, a stimulation pulse is delivered at the selected position using the pulse generator 19. This can be performed manually by the physician or by an attendant personal by inputting a activation instruction using, for example, the keyboard 32 or the screen 33 to the processor 18 instructing it to prompt the pulse generator 19 to deliver a pulse to the cardiac tissue of heart 12 at the position of the electrode 13. Alternatively, the pulse can be delivered on an automatic basis. For example, the pulse generator 19 can be prompted by the processor 18 to deliver the pulse when a user by means of, for example, the keyboard 32 or the screen 33 states that the lead 11 has been placed on a position which shall be evaluated with respect to the heart pumping activity resulting from a stimulation pulse.

Thereafter, at step 54, sensor signals and signals characteristic of the heart activity are recorded. For example, it may be signals indicating the left ventricular and/or right ventricular pressure. That is, physiologic parameters reflecting hemodynamic performance are derived for each lead position. In addition, IEGM signals may also be recorded. As will be discussed in more detail below, there are a number of different parameters that can be used as hemodynamical indicators of the heart pumping activity including the pre-ejection period (PEP), the quotient between PEP and left ventricular ejection time (LVET), the coordination between the contraction of the left ventricle and the contraction of the right ventricle and/or the best proportion between the left ventricular pressure and the right ventricular pressure, the ventricular contractility (peak left ventricular pressure change during systole or LV+dp/dt), or the cardiac output. Subsequently, at step 56, the sensor signals and/or signals characteristic of the heart activity at the selected lead position is stored in the storage means of the evaluation device 15. As will be discussed below, the signal characteristic of the heart activity may depend on which parameter that is used in the evaluation procedure. Furthermore, position data or information of the actual lead position is also obtained and stored in the storage means. As discussed above, this position data may be obtained by means of a number of different methods, for example, X-ray, ultrasound, or by means of the physician by personal feeling from his or hers fingers during the implantation procedure. Preferably, the AV interval between stimulation of the atrium and the ventricle and/or the VV interval between stimulation of the right and left ventricles are optimized before the measurement related to the determination of the performance of the actual lead position is performed.

At step 58, the processor 18 determines a measure or a score value of the heart activity for the actual lead position using the recorded signal data. The measure or score value may be presented for the user at the screen 33, for example, as a numerical value or as a graphical representation. At step 60, the user may select whether another lead position is to be evaluated or tested. If yes, the above mentioned steps 52-58 are repeated. If no, the processing means determines which lead position that results in the most favorable hemodynamics of the heart based on the determined or calculated measure for each lead position at step 62. As will be discussed below, a number of different parameters can be used for this determination. For example, the lead position resulting in the shortest pre-ejection time period (PEP) may be determined to be the optimal site with respect to the hemodynamics of the heart. The measure at each lead position together with the lead position resulting in the most favorable hemodynamics of the heart can be presented for the user visually at the screen 33 of the operator unit 31. Thereby, the user can easily identify the best or optimal position or site for the lead with respect to the desired heart activity in a clear way. These measures can, for example, be presented on the screen 33 by means of a graphical method such as color intensity or a bar diagram.

In the following, different embodiments of the present invention employing different approaches to derive physiologic parameters reflecting hemodynamic performance for different lead positions in order to determine the optimal lead site will be described.

According to one embodiment of the present invention, the pre-ejection period at each lead position is measured and the lead position resulting in the shortest pre-ejection time period (PEP) is determined to be the lead position resulting in the most favorable hemodynamics of the heart. The processor 18 is arranged to determine the PEP as the period from the onset of a QRS or an emitted ventricular stimulation until the opening of the aortic valve. Information regarding the onset of a QRS or an emitted ventricular stimulation is recorded by means of the measuring means 16 and transferred to the processor 18. The sensor 14 includes a sensor arranged to sense the opening of the aortic valve or the ejection of the left ventricle (LV). This may be achieved by measuring variations of the volume, detecting variations in the hemodynamic pressure of the left ventricular, or otherwise detecting the opening of the valve in a reliable manner. In this embodiment, the sensor 14 includes a pressure sensor arranged to detect variations in the pressure within the left ventricular. The pressure sensor is placed tranvenously through the coronary sinus and located in the coronary vein. The pressure that is sensed in this location is proportional to the left ventricular pressure. The design and function of the pressure sensor is described in U.S. Pat. No. 5,129,394, which herein is incorporated by reference in its entirety. During the evaluation procedure, the measuring unit 16 records the signals representative of the pressure variations at each lead position, the signals are stored in the storing unit 17 and transferred to the processor 18, which is arranged to determine the opening of the aortic valve to the moment when a maximum of the time derivative of the ventricular pressure (i.e. dp/dt) occurs after the onset of a QRS complex or an emitted ventricular stimulation. The processor 18 determines the PEP for each lead position, which are used as a measure of the heart activity, where the information regarding the position of the lead is input into the evaluation device 15, for example, by the physician or by attendant personnel via the operator unit 31 as discussed above. As also mentioned above, the results can be presented for the physician and the attendant personnel during the implantation procedure on the screen 33. Then, the processing means 18 is arranged to determine the lead position resulting in the shortest PEP as the lead position resulting in the most favorable hemodynamics of the heart. As PEP tends to increase at, inter alia, cardiac insufficiency, therefore, a correction of the measured PEP with respect to the heart rate may have to be performed.

According to another embodiment of the present invention, the quotient between PEP and left ventricular ejection time (LVET) is determined and the lead position resulting in the lowest value of the quotient between PEP and left ventricular ejection time (LVET) to be the lead position resulting in the most favorable hemodynamics of the heart. The processor 18 is arranged to determine the LVET as the period of time required for the systolic ejection of the left ventricle (LV) and the PEP as the period from the onset of a QRS or an emitted ventricular stimulation until the opening of the aortic valve. LVET depends on the heart rate and must therefore be corrected to it. Generally, LVET is specified as the percentage of the standard value of the heart rate. The standard value of LVET is about 92-108%. LVET is affected by the contractility of the myocardium and by outflow obstructions at the left ventricle. At a degraded contractility with a low stroke volume LVET will decrease, while it will be lengthen at outflow obstructions, such as aortic stenosis, and at a large central stroke volume. PEP, as mentioned above, tends to increase at, inter alia, cardiac insufficiency. As with LVET, PEP must be corrected with respect to the heart rate. The PEP/LVET quotient reflects the function of the left ventricle in a more efficient way than the individual components and is not dependent on the heart rate. The standard value is about 0.30-0.40 for adults. At cardiac insufficiency, the PEP/LVET quotient can increase significantly and may reach values as high as 0.60. In this embodiment, the sensing means 14 includes a pressure sensor placed tranvenously through the coronary sinus and located in the coronary vein. The pressure that is sensed in this location is proportional to the left ventricular pressure. The design and function of the pressure sensor is described in the above mentioned U.S. Pat. No. 5,129,394. Furthermore, the measuring unit 16 records the signals representative of the pressure variations, the signals are stored in the storing unit 17 and transferred to the processor 18, which is arranged to determine the opening of the valve to the moment when a maximum of the time derivative of the ventricular pressure occurs after the onset of a QRS complex or an emitted ventricular stimulation. Information regarding the onset of a QRS or an emitted ventricular stimulation is recorded by means of the measuring unit 16 and transferred to the processor 18. The sensor 14 also includes a sensor for detecting the closing of the aortic valve. This may be achieved by measuring variations of the volume, detecting variations in the hemodynamic pressure of the left ventricular, or otherwise detecting the opening of the valve in a reliable manner. According to this embodiment, the pressure sensor of the sensor 14, which detects variations in the pressure within the left ventricular, is also used to detect the closing of the aortic valve. The measuring unit 16 records the signals representative of the pressure variations, the signals are stored in the storing unit 17 and transferred to the processor 18, which is arranged to determine the closing of the aortic valve to the moment when a maximum of the time derivative of the ventricular pressure (dp/dt) occurs. That is, at the maximum rate of rise of the ventricular pressure. Then, the processor 18 determines the PEP and the LVET for each lead position in order obtain the PEP/LVET quotient for each position, which is used as a measure of the heart activity. The information regarding the position of the lead is input into the evaluation device 15, for example, by the physician or by attendant personnel via the operator unit 31 as discussed above. As also mentioned above, the results can be presented for the physician during the implantation procedure on the screen 33. Finally, the processor 18 is arranged to determine the lead position resulting in the lowest value of the quotient between PEP and LVET to be the lead position resulting in the most favorable hemodynamics of the heart.

According to yet another embodiment of the present invention, the co-ordination of the contraction of the left ventricle and the right ventricle, respectively, or, in other words, the timing difference or delay between the contraction of the left ventricle and the right ventricle and/or the proportion between the left ventricular pressure and the right ventricular pressure is (are) measured at each lead position. The processing means 18 is arranged to determined the lead position resulting in the shortest delay between the contraction of the left ventricle and the contraction of the right ventricle and/or the proportion closest to a predetermined proportion, which predetermined proportion may be selected or defined by the user, between the left ventricular pressure and the right ventricular pressure to be the lead position resulting in the most favorable hemodynamics of the heart. It is known that a lack of synchronism between left and right ventricular side is inefficient and even destructive. In a first approximation synchronous timing is the goal. However, a short delay might be optimal, but that will be shown in clinical studies. Anyhow, the suggested embodiment facilitates processing of measured signals to set an optional timing and/or lead position although a specific optimal value is not known.

In this embodiment, the sensor 14 includes a pressure sensor placed tranvenously through the coronary sinus and located in the coronary vein. The pressure that is sensed in this location is proportional to the left ventricular pressure. The design and function of the pressure sensor is described in the above mentioned U.S. Pat. No. 5,129,394. Moreover, the sensing means of a second lead includes a right ventricular pressure sensor located in the right ventricle arranged to sense variations of the right ventricular pressure. The measuring unit 16 records the signals representative of the pressure variations of the left ventricle and the right ventricle, the signals are stored in the storing unit 17 and transferred to the processor 18. Then, the processing means 18 compares the pressure of the left and right ventricle, respectively, for each lead position in order to determine the co-ordination of the contraction of the left and right ventricle, respectively, and/or the proportionality between the left ventricular pressure and the right ventricular pressure. Accordingly, the delay or timing difference between the contraction of the left ventricle and right ventricle and/or the proportionality between the left ventricular pressure and the right ventricular pressure are determined for each lead position, which are used as a measure of the heart activity, wherein the information regarding the position of the lead is input into the evaluation device 15, for example, by the physician or by attendant personnel via the operator unit 31 as discussed above. As mentioned above, the results can be presented for the physician and the attendant personnel during the implantation procedure on the screen 33. The processor 18 is arranged to determine the lead position resulting in the best co-ordination, i.e. the shortest delay between the contraction of the left ventricle and right ventricle, and/or the best proportionality between the left ventricular pressure and the right ventricular pressure as the lead position resulting in the most favorable hemodynamics of the heart.

According to still another embodiment of the present invention, the ventricular contractility (peak left ventricular pressure change during systole or LV+dp/dt) is measured at each lead position. The processor 18 is arranged to determine the lead position resulting in the highest LV+dp/dt as the lead position resulting in the most favorable hemodynamics of the heart. The left ventricle systolic performance directly determines the ability of the of the heart to pump blood through the systemic circulation and one way to assess the heart activity is to examine how well the left ventricle contracts in order to determine the effectiveness of the left ventricle as a pump. One measure of left ventricle contraction effectiveness is called “contractility”, which is the measure used in this embodiment. The left ventricle contractility is estimated by the processor 18 using the peak positive rate of change of the left ventricular pressure during the systole, i.e. the maximum positive derivative of the left ventricular pressure. It should however be noted that the left ventricular dp/dt max is a widely accepted index of the contractile performance of the left ventricle. In this embodiment, the sensor 14 includes a sensor arranged to detect variations in the pressure within the left ventricular. The pressure sensor is placed tranvenously through the coronary sinus and located in the coronary vein. The pressure that is sensed in this location is proportional to the left ventricular pressure. The design and function of the pressure sensor is described in the above mentioned U.S. Pat. No. 5,129,394, which herein is incorporated by reference in its entirety. It should be noted that there are possible means for estimating the left ventricle systolic performance, for example, by measuring the stroke volume, which is the volume of blood pumped out of the left ventricle per systole. In order to achieve this a sensor for sensing the aortic pulse pressure can be used. During the evaluation procedure, the measuring unit 16 records the signals representative of the pressure variations obtained from the pressure sensor at each lead position, the signals are stored in the storing unit 17 and transferred to the processor 18. The processor 18 determines the LV+dp/dt at each lead position. The information regarding the positions of the lead is input into the evaluation device 15, for example, by the physician or by attendant personnel via the operator unit 31 as discussed above. As also mentioned above, the results can be presented for the physician during the implantation procedure on the screen 33. Then, when LV+dp/dt have been determined for all lead positions, the processor 18 is arranged to determine the lead position resulting in the highest LV+dp/dt value to be the lead position resulting in the most favourable hemodynamics of the heart.

According to a further embodiment of the present invention, the cardiac output is determined at each lead position. The processor 18 is arranged to determine the lead position resulting in the highest cardiac output, which is the volume of blood in litres ejected by the heart per minute, as the lead position resulting in the most favorable hemodynamics of the heart. In this embodiment, a lead 11 is placed in the right ventricle and the sensor 14 includes a right ventricular pressure sensor arranged to sense the pressure in the right ventricle. The measuring unit 16 includes an integrator that integrates the signal corresponding to the sensed pressure between a start time and a stop time to produce a integration result corresponding to the cardiac output. Moreover, the measuring unit 16 includes a filter that filters the pressure signal during a systolic phase to identify the opening of a valve at the right ventricle as the start time for the integration and to identify the closing of this valve as the stop time. The details of the design and function of the right ventricular pressure sensor, the integrator and the filter are described in U.S. Pat. No. 6,314,323, the teachings of which are incorporated herein by reference. During the evaluation procedure, the measuring unit 16 records the cardiac output at each lead position, the results are stored in the storing unit 17 and transferred to the processor 18. The information regarding the positions of the lead is input into the evaluation device 15, for example, by the physician or by attendant personnel via the operator unit 31 as discussed above. As also mentioned above, the results can be presented for the physician during the implantation procedure on the screen 33. Then, when cardiac output has been determined for all lead positions, the processor 18 is arranged to determine the lead position resulting in the highest cardiac output as the lead position resulting in the most favourable hemodynamics of the heart.

According to another embodiment of the present invention, the sensor 14 mounted within the lead 11 includes one or more accelerometer-based cardiac wall motion sensors. The cardiac wall motion sensors transducer accelerations of cardiac tissue to which the lead 11 is placed in close proximity to or attached, so as to provide one or more signals indicative of cardiac mechanical activity. The cardiac wall motion sensor signals are recorded by the measuring unit 16 and are transferred to the processor 18 in which the signals are processed to derive cardiac wall tissues signals including cardiac wall velocity signals and cardiac wall displacement signals. The derived signals are further processed to derive physiologic parameters indicative of cardiac performance, including stroke volume, contractility, pre-ejection period, and ejection time. Preferably, one or more accelerometer-based cardiac wall motion sensors are arranged at the coronary sinus electrode, which is placed in a coronary vein. Thereby, cardiac tissues motion signals indicative of, for example, left ventricular ejection time and pre-ejection period can be collected and recorded for each lead position, and the processor 18 can determine the PEP/LVET quotient for the corresponding lead positions. There can be one accelerometer that is moved to different positions within the heart, evaluation is performed at each position and each position is manually indicated by the operator instead of using a multiaccelerometer catheter. Thereafter, the processor 18 is capable of determining the lead position resulting in the lowest value of the PEP/LVET quotient as the lead position resulting in the most favourable hemodynamics of the heart. The details of the function and design of the sensor and the processing steps to derive the physiologic parameters are described in U.S. Pat. Nos. 5,628,777 and 5,549,650, the teachings of which are incorporated herein by reference. The information regarding the positions of the lead is input into the evaluation device 15 in accordance with the above discussion. As also mentioned above, the results can be presented for the physician during the implantation procedure on the screen 33. In an alternative embodiment, one or more accelerometer-based cardiac wall motion sensors is arranged within a lead placed in the right ventricle in addition to the one or more accelerometer-based cardiac wall motion sensors arranged within lead placed in the coronary vein. Thereby, the contraction of the left ventricle and the right ventricle, respectively, or, in other words, the timing difference or delay between the contraction of the left ventricle and the right ventricle and/or the proportion between the left ventricular pressure and the right ventricular pressure can be measured at each lead position and the processing means can determine the lead position resulting in the best co-ordination, i.e. the shortest delay between the contraction of the left ventricle and right ventricle, and/or the best proportionality between the left ventricular pressure and the right ventricular pressure as the lead position resulting in the most favorable hemodynamics of the heart. As mentioned above, the information regarding the positions of the lead are input into the evaluation device 15, for example, by the physician or by attendant personnel via the operator unit 31 and the results can be presented for the physician during the implantation procedure on the screen 33.

Preferably, the AV interval between stimulation of the atrium and the ventricle and/or the VV interval between stimulation of the right and left ventricles are optimised before the measurement related to the determination of the performance of the actual lead position is performed in the above mentioned different embodiments of the present invention employing different approaches to derive physiologic parameters reflecting hemodynamic performance for different lead positions in order to determine the optimal lead site.

It should be noted that in the different embodiments of the present invention described above, electrical signals, for example, IEGM signals can be used in combination with the signals indicative of the cardiac mechanical activity obtained by means of acoustic sensors, accelerometer-based sensors, or pressure sensors to produce a hemodynamical measure of the lead positions so as to evaluate the different positions in order to identify the optimal position of the lead or the leads.

Although an exemplary embodiment of the present invention has been shown and described, it will be apparent to those having ordinary skill in the art that a number of changes, modifications, or alterations to the inventions as described herein may be made. Thus, it is to be understood that the above description of the invention and the accompanying drawings is to be regarded as a non-limiting example thereof and that the scope of protection is defined by the appended patent claims. As an example, many of the functions described above may be obtained and carried out by suitable software comprise in a micro-chip, an ASIC, or any suitable data carrier.

Although modifications and changes may be suggested by those skilled in the art, it is the intention of the inventors to embody within the patent warranted hereon all changes and modifications as reasonably and properly come within the scope of their contribution to the art.

1. (canceled)

2-50. (canceled)

51. A device for evaluating positions of a medical lead during an implantation procedure, comprising:
a medical lead comprising at least one electrode for electrical stimulating and electrical sensing of in vivo tissue associated with a heart;
a mechanical sensor carried by said lead, said mechanical sensor detecting activity associated with pumping of the heart and emitting a pumping activity signal corresponding thereto;
a measuring unit connected to said medical lead that records an IEGM signal sensed by said at least one electrode and a pumping activity signal sensed by said mechanical sensor, at each of a plurality of different lead positions;
a storage unit that stores, for each of said lead positions, the IEGM signal and the pumping activity signal detected at that lead position; and
a processor having access to said storage unit that determines a lead position, from among said plurality of lead positions, that produces most favorable hemodynamics of the heart, based on said IEGM signal and said pumping activity signal.

52. A device as claimed in claim 51 wherein said mechanical sensor is a pressure sensor that emits a pressure signal as said pumping activity signal.

53. A device as claimed in claim 51 wherein said mechanical sensor is an accelerometer sensor and emits a signal indicative of acceleration as said pumping activity signal.

54. A device as claimed in claim 51 wherein said processor processes said pumping activity signal to derive a physical parameter therefrom indicative of cardiac performance.

55. A device as claimed in claim 51 wherein said processor determines a lead position, as producing said most favorably hemodynamics of the heart, that produces a highest ventricular contractility.

56. A device as claimed in claim 55 wherein said mechanical sensor is a pressure sensor that emits a pressure signal as said pumping activity signal, and wherein said processor determines said highest ventricular contractility as a peak of left ventricular pressure change during systole.

57. A device as claimed in claim 51 wherein said mechanical sensor is a pressure sensor that emits a pressure signal as said pumping activity signal, and wherein said processor determines a lead position, as said lead position producing most favorable hemodynamics, as a lead position that results in a maximum left ventricular pressure.

58. A device as claimed in claim 51 wherein said processor determines a lead position, as said lead position producing most favorable hemodynamics, as a lead position that produces a highest cardiac output.

59. A device as claimed in claim 51 wherein said processor determines a lead position, as said lead position producing most favorable hemodynamics, as a lead position that produces a shortest delay between contraction of the left ventricle and contraction of the right ventricle.

60. A device as claimed in claim 51 wherein said mechanical sensor is a pressure sensor that emits a pressure signal as said pumping activity signal, and wherein said processor determines a lead position, as said lead position producing most favorable hemodynamics, as a lead position that produces a predetermined proportionality between left ventricular pressure and right ventricular pressure.

61. A device as claimed in claim 51 wherein said processor determines a lead position, as said lead position producing most favorable hemodynamics, as a lead position producing a shortest pre-ejection time period.

62. A device as claimed in claim 61 wherein said mechanical sensor comprises an acoustic sensor that senses opening and closing of the aortic mouth, and wherein said processor determines said pre-ejection time period as a time from an onset of a QRS combination in said IEGM or an emitted ventricular stimulation, until opening of the aortic valve is detected.

63. A device as claimed in claim 51 wherein said processor determines a lead position, as said lead position producing most favorable hemodynamics, as a lead position producing a lowest value of a quotient between the pre-ejection time period and left ventricular ejection time.

64. A device as claimed in claim 63 wherein said mechanical sensor is an acoustic sensor that detects opening and closing of the aortic valve, and wherein said processor determines said pre-ejection time period as a time beginning from an onset of a QRS combination in the IEGM or an emitted ventricular stimulation, until opening of the aortic valve is detected, and wherein said processor determines left ventricular ejection time as a time duration required for systolic ejection of the left ventricle.

65. A device as claimed in claim 51 wherein said medical lead is a first medical lead configured for placement in the right ventricle, and wherein said device comprises a second medical lead configured for placement to stimulate the left ventricle, and wherein said device comprises a pulse generator connected to said first and second medical lead that emits stimulation pulses respectively delivered by said first and second medical leads to the right and left ventricles with VV interval therebetween, and wherein said processor optimizes said VV interval before enabling a measurement at the respective lead positions.

66. A device as claimed in claim 51 wherein said medical lead is a first medical lead configured for placement in a ventricle, and wherein said device comprises a second medical lead configured for placement in an atrium, and wherein said device comprises a pulse generator connected to said first and second medical leads for emitting stimulation pulses respectively delivered to the ventricle and the atrium by said first and second medical leads with an AV interval therebetween, and wherein said processor optimizes said AV interval before enabling a measurement at the respective lead positions.

67. A device as claimed in claim 51 wherein said medical lead is a first medical lead configured for placement in the right ventricle, and wherein said device comprises a second medical lead configured for placement in the left ventricle and a third medical lead configured for placement in an atrium, and a pulse generator connected to said first, second, and third medical leads that emits stimulation pulses respectively delivered by said first and second medical leads to the right and left ventricles with a VV interval therebetween, and respectively delivered by one of said first or second medical leads to the right or left ventricle, and by said third medical lead to the atrium, with an AV interval therebetween, and wherein said processor optimizes said AV interval and said VV interval before enabling a measurement at the respective lead positions.

68. A method for evaluating positions of a medical lead during an implantation procedure, said medical lead comprising at least one electrode that stimulates and senses tissue associated with a heart, and said medical lead carrying at least one mechanical sensor that senses pumping activity of the heart and emits a pumping activity signal corresponding thereto, said method comprising the steps of:
recording IEGM signals sensed by said at least one electrode, and pumping activity signals detected by said mechanical sensor, at each of a plurality of different lead positions;
for each of said lead positions, storing the IEGM signal and the pumping activity signal corresponding thereto; and
automatically electronically determining a lead position, from among said plurality of lead positions, producing most favorable hemodynamics of the heart, dependent on the IEGM signals and the pumping activity signal.

69. A method as claimed in claim 68 comprising sensing at least one of right ventricular pressure and left ventricular pressure with said mechanical sensor, and emitting a pressure signal as said pumping activity signal.

70. A method as claimed in claim 68 comprising determining a lead position, as said lead position producing most favorably hemodynamics, as a lead position resulting in a highest ventricular contractility.

71. A method as claimed in claim 68 comprising determining a lead position, as said lead position producing most favorably hemodynamics, as a lead position resulting in a maximum left ventricular pressure.

72. A method as claimed in claim 68 comprising determining a lead position, as said lead position producing most favorably hemodynamics, as a lead position resulting in a highest cardiac output.

73. A method as claimed in claim 68 comprising determining a lead position, as said lead position producing most favorably hemodynamics, as a lead position resulting in a shortest pre-ejection time.

74. A method as claimed in claim 68 comprising determining a lead position, as said lead position producing most favorably hemodynamics, as a lead position resulting in a lowest quotient between the pre-ejection time period and left ventricular ejection time.

75. A computer-readable medium encoded with a data structure for use with an implantable medical device during an implantation procedure having a medical lead comprising at least one electrode for electrical stimulating and sensing of tissue associated with a heart, and at least one mechanical sensor carried by the medical lead that detects signals indicative of pumping activity of the heart, said device being operated by a processor and said data structure causing said processor to:
initiate recording IEGM signals sensed by said at least one electrode, and pumping activity signals detected by said mechanical sensor, at each of a plurality of different lead positions;
store said IEGM signals and said pumping activity signals for each lead position; and
automatically calculate a lead position, from among said plurality of lead positions that produces a most favorable hemodynamics of the heart.
